(19) United States
(12) Patent Application Publication
(10) Pub. No.: US 2011/0244035 A1
(43) Pub. Date: Oct. 6, 2011

(54) Dosage Forms

(76) Inventors: Chin-Ming Chang, Tustin, CA (US); James N. Chang, Newport Beach, CA (US); E. Quinn Farnes, Laguna Beach, CA (US)

(21) Appl. No.: 12/939,472

(22) Filed: Nov. 4, 2010

Related U.S. Application Data
(63) Continuation of application No. 11/676,577, filed on Feb. 20, 2007, now abandoned.
(60) Provisional application No. 60/776,483, filed on Feb. 24, 2006.

(51) Int. Cl. A61K 9/48; A61K 9/14; A61K 31/4439; C07D 401/12; A61K 9/00; A61P 1/00; A61J 1/03

(52) U.S. Cl. 424/456; 424/490; 514/338; 424/497; 424/451; 546/273.7; 424/400; 206/534

(57) ABSTRACT

Dosage forms comprising therapeutically active compounds are disclosed herein.

### CROSS-REFERENCE TO RELATED APPLICATION

This application is based, and claims priority under 35 U.S.C. §120 to U.S. Provisional Patent Application No. 60/776,483 filed on Feb. 24, 2006, and which is incorporated herein by reference.

### DESCRIPTION OF INVENTION

### Brief Description of the Invention

Disclosed herein is a dosage form comprising beads, wherein each bead consists of:

a sugar sphere forming a core of said bead,
six layers consisting of three drug layers and three seal film layers alternating from said core,
wherein a drug layer is the first layer coating said sugar sphere and wherein a seal film layer covers the other five alternating layers, and an enteric coat;
wherein each seal film layer comprises a sealer; and
wherein each drug layer comprises a compound, a pharmaceutically acceptable salt of a compound, or a combination thereof, said compound having a formula chosen from

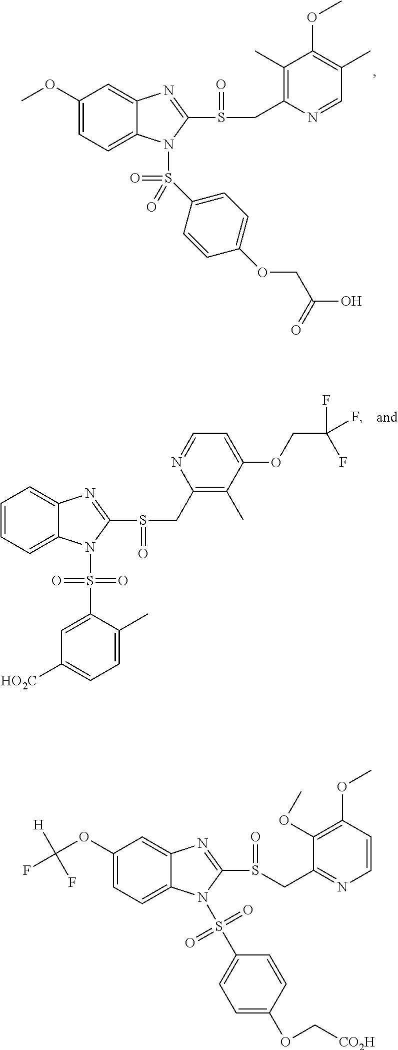

### BRIEF DESCRIPTION OF THE DRAWINGS

FIG. 1 illustrates a typical dosage form.

FIG. 2 outlines a procedure for preparing a dosage form.

### DETAILED DESCRIPTION OF THE INVENTION

A sugar sphere is known in the art for use as a core of a dosage form. Any type of sugar which is solid under the process conditions may be used. For example, monosaccharides such as glucose, galactose, mannose, fructose, and the like; and disaccharides such as sucrose, lactose, maltose and the like, are useful.

A drug layer is a layer that comprises the drug. The drug layer may also contain one or more excipients.

In one dosage form each drug layer consists of a drug and a sealer.

In another dosage form each drug layer consists of said drug, hydroxypropylmethylcellulose, and polyethylene glycol 400.

A seal film layer is a layer comprising a sealer that coats.

To coat is to form a substantially complete layer of a material over another underlying substance or layer. Coating is synonymous to “to coat” or coating may also be the layer formed.

A sealer is a cohesive material that is capable of forming a coating either by itself or in combination with other materials. Suitable sealers include, for example, celluloses such as hydroxypropylmethylcellulose, hydroxypropyl cellulose, microcrystalline cellulose and carboxymethyl-cellulose sodium, polyvinyl pyrrolidone, polyethylene glycol, starches, and combinations thereof.

In another dosage form the sealer consists of hydroxypropylcellullose and polyethylene glycol.

In another dosage form the sealer consists of hydroxypropylmethylcellulose and polyethylene glycol 400.

In another dosage form the sealer is Opadry® clear.

An enteric coat is a coat that is made of a material that prevents the drug from being released in the lower pH areas of the gastrointestinal tract, but allows release as the dosage form moves to the higher pH areas of the gastrointestinal tract. Enteric coats may be applied by a number of traditional methods including, but no limited to, conventional coating procedures or fluid bed spraying. Suitable enteric coating materials include one or more polymers. Examples include methacrylic acid copolymers, cellulose acetate butyrate, cellulose acetate trimellitate, carboxymethylethylcellulose, shellac, Eudragit L, Eudragit S (Rohm Pharma), hydroxypropyl methylcellulose acetate succinate, hydroxypropyl methylcellulose phthalate, polyvinyl acetate phthalate, cellulose acetate phthalate, and the like. Eudragit L and Eudragit S are tradenames of co-polymerized methacrylic acid/methacrylic acid methyl esters.

The enteric coat may contain pharmaceutically acceptable plasticizers to obtain desirable mechanical properties, such as flexibility and hardness of the enteric coating layers. Such plasticizers are for instance, but not restricted to, triacetin, citric acid esters, triethyl citrate, phthalic acid esters, diethyl phthalate, dibutyl sebacate, cetyl alcohol, polyethylene glycols, polysorbates or other plasticizers. Additives such as dispersants, colorants, pigments, polymers e.g. poly(ethylacrylate, methylmethacrylate), anti-tacking and anti-foaming agents may also be included in the enteric coat. Other compounds may be added to increase film thickness and to decrease diffusion of acidic gastric juices into the acidic susceptible active substance.

In another dosage form the enteric coat consists of triethyl citrate, talc, and methacrylic acid copolymer.

In another dosage form the enteric coat consists of about 54 mg of methacrylic acid copolymer, about 13.5 mg of talc, and about 5.5 mg of triethyl citrate.

Unless otherwise indicated, % is intended to mean % (w/w) in the specification and claims herein.

In another dosage form said beads consist essentially of about 35.5% drug, 33.5% sugar sphere, 14.8% methacrylic acid copolymer, 11% Opadry® clear, 3.7% talc, and 1.5% triethyl citrate; and wherein said beads are in a gelatin capsule.

In another dosage form said beads consist of about 122 mg of the sugar spheres, about 73 mg of the enteric coat, and about 40 mg of the binder layers.

Another dosage form has from about 120 mg to about 130 mg of drug, said drug consisting of

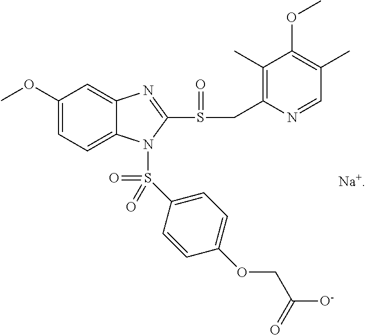

The beads disclosed herein are used to fill a capsule to make a dosage form. The amount of active in the capsule depends on the size of the capsule as depicted in Table 1 below.

TABLE 1

| Capsule Size | Active Content (mg) |
|---|---|
| 00 | 170-300 |
| 0 | 130-240 |
| 1 | 100-180 |
| 2 | 75-135 |
| 3 | 60-100 |
| 4 | 50-75 |

In a situation where there are three drug layers and three seal film layers alternating from the core, the following layers exist, going outward radially from the core-drug layer (1), seal film layer (1), drug layer (2), seal film layer (2), drug layer (3), seal film layer (3). Seal film layer (3) is covered by the enteric coat.

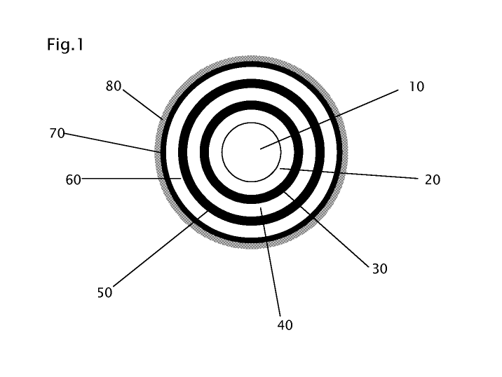

FIG. 1 illustrates a typical dosage form, the core (10) is in the center of the dosage form. A first drug layer (20) is the next layer from the core. A first seal film layer (30) is the next layer from the core. Two more drug (40, 60) and seal film (50, 70) then alternate, and the enteric coat (80) covers the entire dosage form.

Another dosage form consists of a plurality of beads in a capsule, said dosage form comprising about 120 mg to about 130 mg of a drug, wherein said drug is sodium {4-[5-methoxy-2-(4-methoxy-3,5-dimethyl-pyridin-2-ylmethanesulfinyl)-benzoimidazole-1-sulfonyl]-phenoxy}-acetate;

wherein each bead consists of
a sugar sphere forming a core,
three drug layers, and
three binder layers; and
an outer layer of said beads consisting of an enteric coat;
wherein said drug layers and said binder layers alternately coat one another, a drug layer being the first layer on the core and a binder layer being the last of the layers that alternately coat one another.

In another dosage form said drug layers consist of the drug and a binder.

In another dosage form said drug layers consist of the drug, hydroxypropylmethylcellulose, and polyethylene glycol 400.

A binder layer is a layer designed to improve the structural strength of the dosage form. A binder layer comprises one or more binders, which are substances which are useful in holding other excipients or active ingredients together as solids. Suitable binders include, for example, celluloses such as hydroxypropylmethylcellulose, hydroxypropyl cellulose, microcrystalline cellulose and carboxymethyl-cellulose sodium, polyvinyl pyrrolidone, polyethylene glycol, starches, and combinations thereof.

In another dosage form said binder layers consist of hydroxypropylcellullose and polyethylene glycol.

In another dosage form said binder layers consist of hydroxypropylmethylcellulose and polyethylene glycol 400

In another dosage form the binder is Opadry® clear.

In another dosage form the enteric coat consists of triethyl citrate, talc, and methacrylic acid copolymer.

In another dosage form the enteric coat consists of about 54 mg of methacrylic acid copolymer, about 13.5 mg of talc, and about 5.5 mg of triethyl citrate.

In another dosage form the plurality of beads consist of about 35.5% drug, 33.5% sugar sphere, 14.8% methacrylic acid copolymer, 11% Opadry® clear, 3.7% talc, and 1.5% triethyl citrate; and wherein said beads are in a gelatin capsule.

In another dosage form the plurality of beads consist of about 122 mg of the sugar spheres, about 73 mg of the enteric coat, and about 40 mg of the binder layers.

Another dosage form has from about 120 mg to about 130 mg of drug, said drug consisting of

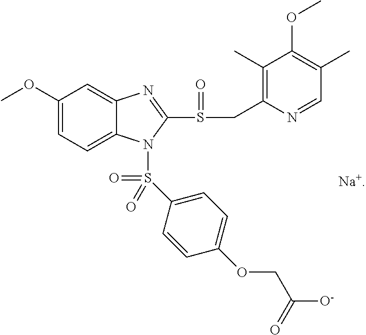

In another embodiment, the dosage form has from about 170 mg to about 300 mg of drug, said drug consisting of

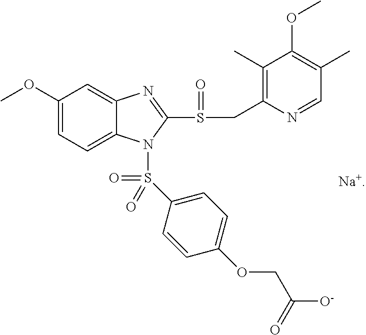

In another embodiment, the dosage form has from about 130 mg to about 240 mg of drug, said drug consisting of

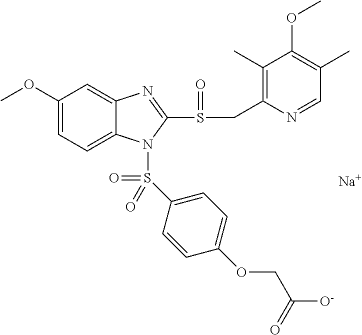

In another embodiment, the dosage form has from about 100 mg to about 180 mg of drug, said drug consisting of

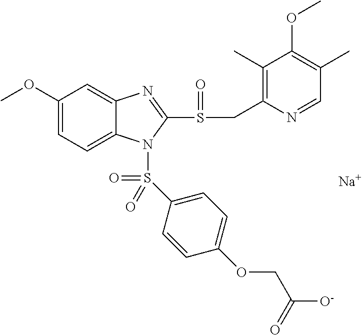

In another embodiment, the dosage form has from about 75 mg to about 135 mg of drug, said drug consisting of

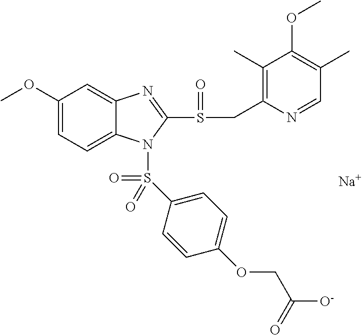

In another embodiment, the dosage form has from about 60 mg to about 100 mg of drug, said drug consisting of

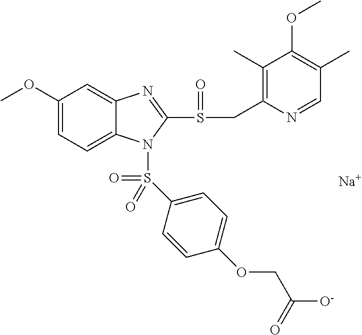

In another embodiment, the dosage form has from about 50 mg to about 75 mg of drug, said drug consisting of

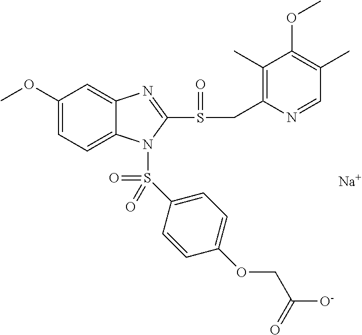

Layers which alternatively coat one another form alternating layers passing radially through the dosage form.

Another dosage form has about 120 mg to about 130 mg of sodium {4-[5-methoxy-2-(4-methoxy-3,5-dimethyl-pyridin-2-ylmethanesulfinyl)-benzoimidazole-1-sulfonyl]-phenoxy}-acetate.

Another dosage form has about 120 mg to about 130 mg of sodium {4-[5-methoxy-2-(4-methoxy-3,5-dimethyl-pyridin-2-ylmethanesulfinyl)-benzoimidazole-1-sulfonyl]-phenoxy}-acetate and further comprises an enteric coat.

A kit is contemplated for each of the foregoing dosage forms or embodiments of dosage forms. The kit comprises the drug a package, and instructions indicating once a day oral administration of said dosage form.

In one embodiment, the kit comprises a dosage form having about 120 mg to about 130 mg of sodium {4-[5-methoxy-2-(4-methoxy-3,5-dimethyl-pyridin-2-ylmethanesulfinyl)-benzoimidazole-1-sulfonyl]-phenoxy}-acetate, a package, and instructions indicating once a day oral administration of said dosage form.

In another embodiment, the dosage form in the kit further comprises an enteric coat. The dosage forms disclosed herein are useful for treatment of gastric conditions, particularly those related to gastric acid secretion.

A person of ordinary skill in the art can determine the dosage regime used for the dosage forms disclosed herein. In one treatment, the person receives one dosage form a day, although 2 or more dosage forms may be used. Dosage forms may also be administered less than once a day, such as every other day, or weekly.

### Example 1

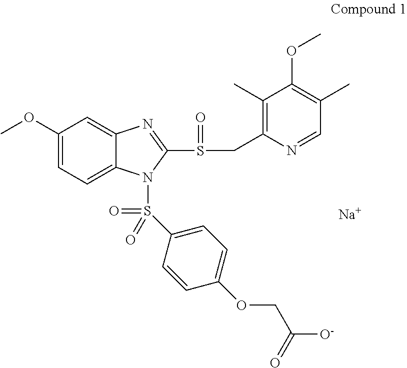

TABLE 2

| | | | Estimated |
|---|---|---|---|
| | | | Amount in |
| | | % | Capsule |
| Ingredient | Function | w/w | (mg) |
| | | 35.5 | 129.6 |
| Sugar Spheres NF (Mesh 20-25) | Core Material | 33.5 | 122.3 |
| Methacrylic Acid Copolymer | Enteric Coat | 14.8 | 54.0 |
| Dispersion NF | | | |
| Opadry ® Clear | Binder/Sealer | 11.0 | 40.2 |
| Talc USP | Glidant | 3.7 | 13.5 |
| Triethyl Citrate NF | Plasticizer | 1.5 | 5.5 |
| Fill Weight | | 100% | 365 |
| Natural Transparent Hard Shell | Dosage | Size | 77.1 |
| Gelatin Capsules | Carrier | #1 | |
| Total Capsule Weight | | | 442.1 |

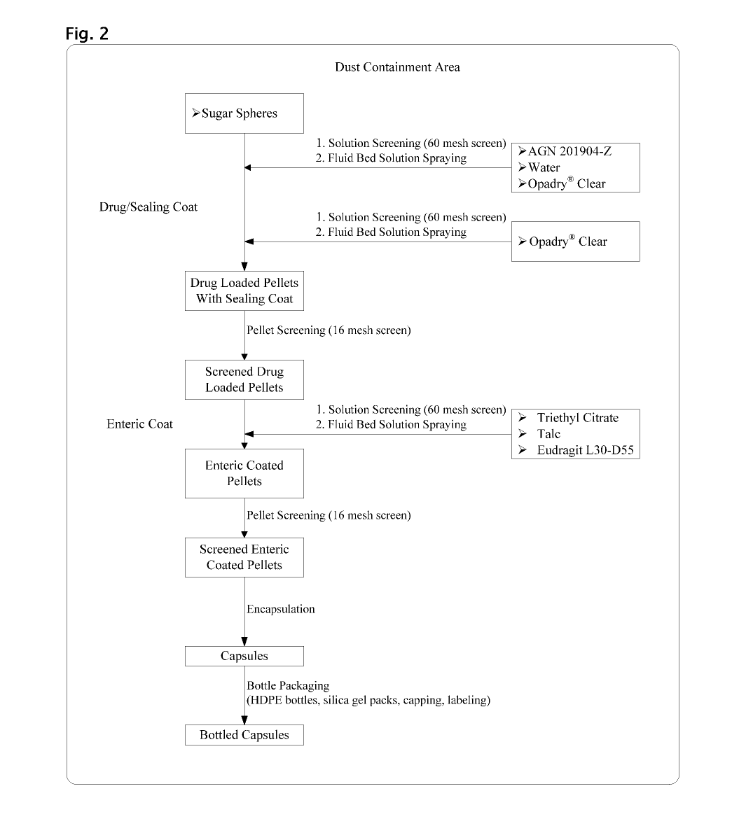

A dosage form having the composition of Table 2 and generally structured as depicted in FIG. 1, is prepared by the procedure outlined in FIG. 2.

### Batch Size Calculation:

| Quantity | Unit | Component |
|---|---|---|
| 240 | g | Compound 1 |
| 58 | g | Opadry ® |
| 220 | g | Sugar spheres |
| 518 | g | Total Weight (no loss) |

### 1. Drug Application

Step 1. Prepare a 10% Opadry® solution using 58 g Opadry®, and 450 mL water. Mix until dissolved (overnight at room temperature is best).
Step 2. To a beaker add 450 mL water and 240 g Compound 1. Mix until dissolved (approximately 30 minutes), then add 27 mL 0.5N HCl. Allow the solution to mix ˜24 hours based on projected middle of spray time.
Step 3. To the above, add 10% Opadry® solution prepared in Step 1. Continue mixing for 10 minutes. Pass the solution through #60 mesh stainless steel sieve to remove any undissolved material (there should be none)

### 2. Prepare Sealing Coat

Step 1. Prepare 5% Opadry® solution for sealing coat by mixing 16.0 g Opadry® and 260 mL water until dissolved. Avoid foaming. Pass through #60 mesh sieve into a clean beaker.

### 3. Apply Drug/Seal Coat in Layers:

Note: it is okay to spray up to 4.5 mL/minute.
Step 1. Spray 550 mL of the Drug/Opadry® solution prepared in #1, Step 3 on 220 g #2025 sugar beads.
Step 2. Spray 50 mL of the Opadry® sealing solution prepared in #2 Step 1 onto the beads.
Step 3. Spray an additional 300 mL of the Drug/Opadry® solution onto the beads.
Step 4. Spray an additional 50 mL of the Opadry® sealing solution onto the beads.
Step 5. Spray the remaining Drug/Opadry® solution onto the beads.
Step 6. Spray the remaining Opadry® sealing solution onto the beads.
Dry for 10 minutes at 55 C inlet temperature. Weigh coated beads, and store overnight if necessary in a screw-capped glass or plastic container with desiccant packets.

### Enteric Coating Calculation

### Step 1. Quantities of Ingredients for Enteric Coating

| | | |
|---|---|---|
| 100 | g | Weight of Eudragit L 30 D-55 polymer needed |
| 333.3 | mL | Volume of Eudragit L 30 D-55 polymer to use (30% solids) |
| 25.0 | g | Weight of talc to use |
| 10.0 | g | Weight of TEC to use |
| 8.8 | mL | Volume of TEC to use (density = 1.137) |
| 332.9 | mL | Volume of water to add |

### Enteric Coating Preparation

Step 1. To a beaker, add 332.9 mL water, 8.8 mL TEC, and 25 g Talc. Mix using high-shear mixer for 10 minutes.
Step 2. Begin mixing gently using low-shear overhead propeller mixer.

To the contents of the beaker gently add 333.3 mL Eudragit L 30 D-55 Polymer. Continue mixing gently 10 minutes. Adjust mixer so no bubbles form. Pass solution through #60 mesh stainless steel sieve to remove any undissolved material (there may be some).

The enteric coating solution can now be sprayed on the beads. Spray starting at 3 mL/min, increase to 4.0 ml/min within 30-45 minutes. Increase to 4.5 ml/min after 1 hour. When finished, dry to 0.6% moisture as before.

What is claimed is:

1. A dosage form comprising beads, wherein each bead consists of:
a sugar sphere forming a core of said bead,
six layers consisting of three drug layers and three seal film layers alternating from said core,
wherein a drug layer is the first layer coating said sugar sphere and wherein a seal film layer covers the other five alternating layers, and
an enteric coat;
wherein each seal film layer comprises a sealer; and
wherein each drug layer comprises a compound, a pharmaceutically acceptable salt of a compound, or a combination thereof, said compound having a formula chosen from,

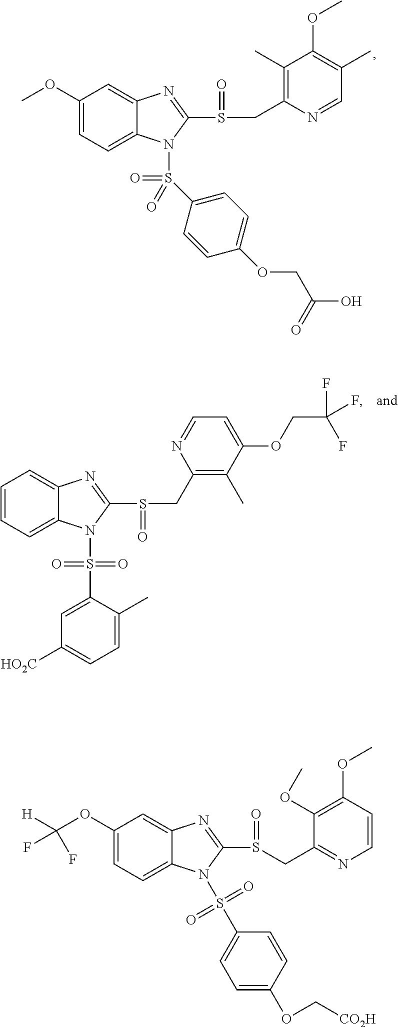

2. The dosage form of claim 1 wherein each drug layer consists of a drug and a sealer.

3. The dosage form of claim 2 wherein the sealer consists of a combination of hydroxypropylcellullose and polyethylene glycol.

4. The dosage form of claim 3 wherein the enteric coat consists of triethyl citrate, talc, and methacrylic acid copolymer.

5. The dosage form of claim 4 wherein the sealer is Opadry® clear.

6. The dosage form of claim 5 wherein said beads consist essentially of about 35.5% drug, 33.5% sugar sphere, 14.8% methacrylic acid copolymer, 11% Opadry® clear, 3.7% talc, and 1.5% triethyl citrate; and wherein said beads are in a gelatin capsule.

7. The dosage form of claim 1 having from about 120 mg to about 130 mg of drug, said drug consisting of

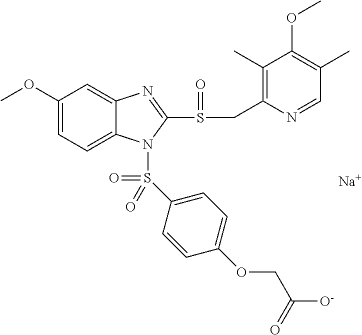

8. A dosage form consisting of a plurality of beads in a capsule, said dosage form comprising about 120 mg to about 130 mg of a drug, wherein said drug is sodium {4-[5-methoxy-2-(4-methoxy-3,5-dimethyl-pyridin-2-ylmethanesulfinyl)-benzoimidazole-1-sulfonyf]-phenoxy}-acetate; wherein each bead consists of a sugar sphere forming a core,
three drug layers, and
three binder layers;
an outer layer of said beads consisting of an enteric coat;
wherein said drug layers and said binder layers alternately coat one another, a drug layer being the first layer on the core and a binder layer being the last of the layers that alternately coat one another.

9. The dosage form of claim 8 wherein said drug layers consist of said drug, hydroxypropylmethylcellulose, and polyethylene glycol 400.

10. The dosage form of claim 8 wherein said binder layers consist of hydroxypropylmethylcellulose and polyethylene glycol 400.

11. The dosage form of claim 8 wherein the plurality of beads consist of about 122 mg of the sugar spheres, about 73 mg of the enteric coat, and about 40 mg of the binder layers.

12. The dosage form of claim 11 wherein the enteric coat consists of about 54 mg of methacrylic acid copolymer, about 13.5 mg of talc, and about 5.5 mg of triethyl citrate.

13. A dosage form having from about 120 mg to about 130 mg of sodium {4-[5-methoxy-2-(4-methoxy-3,5-dimethyl-pyridin-2-ylmethanesulfinyl)-benzoimidazole-1-sulfonyl]-phenoxy}-acetate.

14. The dosage form of claim 13 which further comprises an enteric coat.

15. A kit comprising a dosage form having from about 120 mg to about 130 mg of sodium {4-[5-methoxy-2-(4-methoxy-3,5-dimethyl-pyridin-2-ylmethanesulfinyl)-benzoimidazole-1-sulfonyl]-phenoxy}-acetate, a package, and instructions indicating once a day oral administration of said dosage form.

16. The kit of claim 15 wherein the dosage form further comprises an enteric coat.

17. The dosage form of claim 1 having from about 170 mg to about 300 mg of drug, said drug consisting of

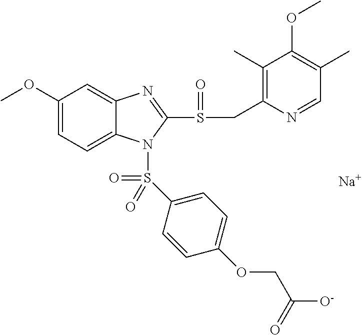

18. The dosage form of claim 1 having from about 130 mg to about 240 mg of drug, said drug consisting of

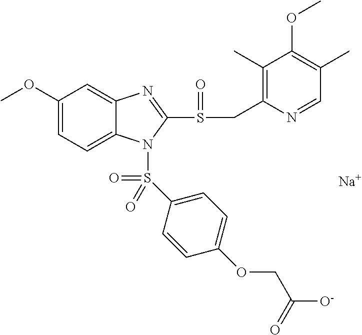

19. The dosage form of claim 1 having from about 100 mg to about 180 mg of drug, said drug consisting of

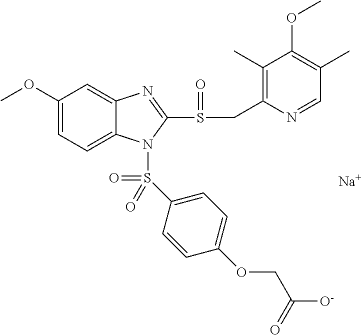

20. The dosage form of claim 1 having from about 75 mg to about 135 mg of drug, said drug consisting of

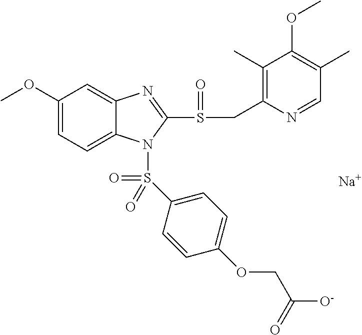

21. The dosage form of claim 1 having from about 60 mg to about 100 mg of drug, said drug consisting of

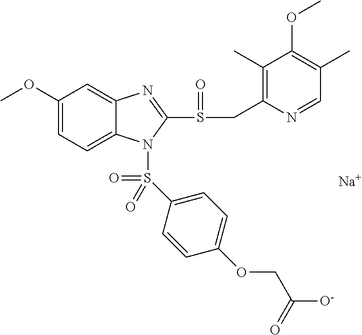

22. The dosage form of claim 1 having from about 50 mg to about 75 mg of drug, said drug consisting of [chemical structure]
